(19) United States
(12) Patent Application Publication
(10) Pub. No.: US 2005/0060020 A1
(43) Pub. Date: Mar. 17, 2005

(54) Covered stent with biologically active material

(75) Inventor: Mark L. Jenson, Greenfield, MN (US)

Correspondence Address: HOFFMANN & BARON, LLP, 6900 JERICHO TURNPIKE, SYOSSET, NY, 11791, US

(73) Assignee: SCIMED Life Systems, Inc.

(21) Appl. No.: 10/664,454

(22) Filed: Sep. 17, 2003

(51) Int. Cl. A61F 2/06

(52) U.S. Cl. 623/1.13; 623/1.42; 623/1.44

(57) ABSTRACT

A composite device provides for the delivery of bioactive agents associated therewith. The device includes a first polymeric liner and a second polymeric liner bonded to the first liner. The device further includes an intermediate structural member interposed between the liner, which is defined by solid segments and openings between the solid segments. The liners are joined through the openings to form at least one pocket about the solid segments. A bioactive agent is located within the pockets about the solid segments of the intermediate structural member.

### FIELD OF THE INVENTION

The present invention relates to an implantable prosthesis for the delivery of a bioactive material to the site of implantation. In particular, the present invention relates to a composite intraluminal device including a structural member, which can be a stent, having on its luminal side a polymeric liner and on its opposite side another polymeric liner. The liners are joined to form a reservoir pocket for containing a bioactive agent associated with the device.

### BACKGROUND OF THE INVENTION

It is well known to implant a stent within a blood vessel to open and/or reinforce collapsing or partially occluded sections of the vessel. Often, stents may be used in conjunction with a graft which provides additional support for blood flow through weakened sections of the blood vessel.

Stents generally are open-ended and are radially expandable between a generally unexpanded insertion diameter and an expanded implantation diameter which is greater than the unexpanded insertion diameter. Stents are often flexible in configuration, which allows them to be inserted through and conform to tortuous pathways in the blood vessels. The stent is generally inserted in a radially compressed state and expanded either through a self-expanding mechanism, or through the use of balloon catheters. For example, various stents and their method of deployment are shown in U.S. Pat. No. 4, 4,503,569 to Dotter; 4,733,665 to Palmaz; U.S. Pat. No. 4,856,561 to Hillstead; U.S. Pat. No. 4,580,568 to Gianturco; U.S. Pat. No. 4,732,152 to Wallsten and 4,886,062 to Wiktor. Published PCT WO 96/03092 A1, based on U.S. priority application Ser. Nos. 08/282,181 and 08/457,354, discloses a tubular shaped stent which is inflatable by a balloon and which shrinks minimally in the longitudinal direction during expansion. The foregoing WO publication and its U.S. priority applications, and the aforementioned U.S. patents are incorporated herein by reference. Additionally, published PCT WO 96/26689, entitled “Improved Longitudinally Flexible Expandable Stent” and being based on U.S. priority application Ser. Nos. 08/396,569 filed Mar. 1, 1995 and Ser. No. 08/511,076 filed Aug. 3, 1995, also discloses stents useful in the present invention, both the WO publication and its U.S. priority application being incorporated by reference herein.

In vascular applications, grafts, stents and stent-graft composites are manufactured from expanded polytetrafluoroethylene (ePTFE) tubes. Extruded PTFE tubes having minimal wall thickness are described in commonly owned, copending U.S. application Ser. No. 10/012,919. An apparatus and method for extrusion of thin-walled PTFE tubes are described in commonly owned, copending U.S. application Ser. No. 10/012,825.

Both textile and polymeric grafts (i.e. PTFE grafts), when used alone suffer from kinking and radial collapse subsequent to implantation. Moreover, when stents are used alone, that is without a graft, patency of the vessel is well maintained, but the problems of excessive cell growth through the stent, as well as thrombis formation and plaque build-up are associated therewith. For these reasons, in endovascular applications, the use of graft/stent combinations has become increasingly important. In particular, by combining a graft with a stent in a composite structure, one gains the advantages of the relatively smooth fluid-contacting surfaces of a graft with the structural support advantages of a stent.

One approach used to attach a graft made from ePTFE material to a stent is disclosed in U.S. Pat. No. 6,139,573, the entire contents of which is herein incorporated by reference. This patent discloses attaching ePTFE material to a stent by using an anchoring material which can be carried into and entrapped in the porous surface of ePTFE. In particular, the patent discloses that an outer stent covering is adhered or otherwise affixed to an inner stent covering, i.e. a liner, at a location substantially coextensive with the inner stent surface. The invention also teaches adhering the outer stent covering to the inner stent covering so as to maintain an air gap therebetween adjacent the stent structure so as to provide domains of relatively high porosity for promoting neointima in-growth.

Attempts to increase the radial tensile and axial tear strengths of microporous ePTFE tubes include forming the tubular grafts of multiple layers placed over one another. Examples of multi-layered ePTFE tubular structures useful as implantable prostheses are shown in U.S. Pat. Nos. 4,816,339; 4,478,898; 5,001,276; 5,800,512; 5,749,880; 5,810,870; and 5,824,050.

It is further known to provide a tubular vascular graft of ePTFE with layers sufficient to provide a differential cross-section of permeability and/or porosity to achieve enhanced healing and tissue in-growth. For example, U.S. Pat. No. 5,800,512 describes a multi-layered ePTFE composite tubular structure including a tissue contacting expanded outer tube and a concentrically adjacent expanded inner tube, an inner surface if which is a blood contacting surface. The graft has an inner tube with an IND of greater than 40 microns and an outer tube of ePTFE having an IND of less than 40 microns. Moreover, U.S. Pat. No. 5,824,050 discloses a multi-layered tubular graft, which may be formed of layers of ePTFE having different porosities.

It is also known to incorporate therapeutic agents into implantable ePTFE materials. The use of therapeutic agents in ePTFE prosthetics is desirable to prevent various complications which may arise as a result of implantation of the prosthetic and to promote cell endothelization, tissue in-growth, and healing. Such therapeutic agents can be provided in the ePTFE material as a dispersion in a biocompatible, biodegradable material. Various pharmacological active agents, such as anti-microbials, anti-virals, antibiotics, growth factors, and blood clotting modulators such as heparin, can be added to the material such that these agents are introduced into the body as the material is bioresorbed. For example, U.S. Pat. No. 5,665,114 to Weadock, et al. discloses and implantable ePTFE prosthesis which incorporates a biocompatible, biodegradable material of natural origin.

U.S. Pat. No. 5,411,550 also describes an implantable prosthetic device for delivering a bioactive material into a blood vessel of a patient. The device includes a single tubular body of ePTFE extruded as a continuous wall, the wall having at least a primary and secondary lumen, wherein the secondary lumen receives the bioactive material. A disadvantage of this device is that because the tubular body is extruded as a single continuous wall, it is not possible to provide a luminal surface and a tissue contacting surface with distinct porosities.

Copending U.S. application Ser. No. 09/962,062 describes an implantable composite device for regulating delivery of bioactive agents associated with the device to a site of implantation. The device includes ePTFE layers of different porosities and may include a reservoir within the ePTFE layer for containing a drug.

There is a need to provide additional ePTFE stent/graft configurations which provide for delivery of therapeutic agents incorporated therein to a site of implantation of the device, and which desirably exhibit distinct porosities at each of the luminal and tissue contacting surfaces.

### SUMMARY OF THE INVENTION

The present invention provides for an implantable composite device for delivery of bioactive agents associated therewith to a site of implantation of the device. The device includes a first polymeric liner; a second polymeric liner; and an intermediate structural member interposed between the first and second polymeric liners. The intermediate structural member is defined by solid segments and openings therebetween such that the first liner can be bonded to the second liner through the openings to form at least one pocket about the solid segments. The device further includes a bioactive agent located within the pocket about the solid segments of the intermediate structural member.

The invention also provides for a device that includes an elongate stent having a generally cylindrical tubular body defined by solid segments and openings between the solid segments. The tubular body defines an inner surface and an opposed outer surface. A first polymer liner is positioned about the inner surface of the tubular body and a second polymer liner is positioned about the outer surface of the tubular body. The second polymer liner is joined to the first liner through the stent openings to form a pocket about the solid segments. A bioactive agent is located within the pocket about the solid segments of the tubular body.

In another aspect of the invention there is provided a method of making an implantable composite device for delivery of bioactive agents associated therewith to a site of implantation of the device. The method includes the steps of: providing a first polymeric liner; providing a second polymeric liner; and interposing an intermediate structural member between the first and second polymeric liners, the intermediate structural member being defined by solid segments and openings therebetween. The method also includes the steps of: joining the first and second polymeric liners through the openings between the solid segments to form reservoir pockets adjacent to the solid segments; and filling the reservoir pockets with a bioactive agent for delivery of the bioactive agent to the site of implantation of the device.

Another method of making the device is provided, where the intermediate structural member between the liners is specifically a stent. This method includes providing an implantable prosthetic stent having a generally cylindrical tubular body defined by solid segments and spaces therebetween, the tubular body defining an inner surface and an opposed outer surface; applying a first polymer liner to the inner surface and applying a second polymer liner to the outer surface. The method further includes the steps of joining the first and the second polymer liners through the spaces between the stent segments to form a reservoir pocket adjacent to the stent segments; and filling the reservoir pocket with a bioactive agent for delivery of the bioactive agent to the site of implantation of the device.

Furthermore, a method for treating a lumen in a body is provided. The method including the steps of: inserting a generally cylindrical implantable composite device for delivery of bioactive agents incorporated therewith into the lumen and affixing the implantable composite device to said lumen such that it will stay where positioned. The device which is inserted includes a first polymeric liner; a second polymeric liner; and an intermediate structural member interposed between the first and second liners. The intermediate structural member of the device is defined by solid segments and openings therebetween such that the first liner can be bonded to the second liner through the openings to form at least one pocket about the solid segments. The composite device for treating a lumen in a body further includes a bioactive agent located within said pocket.

The present invention also provides a method for treating a body lumen with a stent-graft. This method includes inserting the stent-graft device for delivery of bioactive agents associated therewith into the body lumen. The device which is inserted includes: an elongate stent having a generally cylindrical tubular body defined by solid segments and openings between the solid segments, the tubular body defining an inner surface and an opposed outer surface; a first polymeric liner positioned about the inner surface of the tubular body; a second polymeric liner positioned about the outer surface of the tubular body; the second polymeric liner being joined to the first liner through the stent openings to form a pocket about the solid segments; and a bioactive agent located within the pocket about the solid segments of the tubular body. This treatment method also includes affixing the stent-graft device to the body lumen such that it will stay where positioned.

### BRIEF DESCRIPTION OF THE DRAWINGS

FIG. 1 is an exploded perspective view of an assembled composite endoprosthesis of the present invention.

FIGS. 2 and 3 are cross-sectional views of an embodiment of the present invention showing the pocket for containing a bioactive agent that is formed by the joining of the outer tubular liner and inner tubular liner enclosing the stent.

FIG. 4 is a cross-sectional view of the device of FIG. 2 following incorporation of a bioactive agent within the pocket.

FIG. 5 is a cross-sectional view of another embodiment of the device of FIG. 2 following incorporation of a fluid mixture that includes an encapsulated bioactive agent.

FIG. 6 is a cross-sectional view of an embodiment of the device of FIG. 4 wherein the porosity of the inner tubular liner is different from the porosity of the outer tubular liner enclosing the stent.

FIG. 7 is a cross-sectional view of a vascular graft of the present invention that includes an intermediate structural element defined by foreign bodies and openings therebetween.

### DETAILED WRITTEN DESCRIPTION

The present invention contemplates adhering, laminating, or otherwise bonding a fusable polymeric layer on either side of an open intermediate component and fusing the layers together to form a reservoir pocket for containing a bioactive agent therewithin. As used herein, the term “bioactive agent” is intended to include any therapeutic agents, diagnostic agents, prognostic agents, or any combination thereof. It is contemplated that the fusion of the polymeric layers may be achieved by various techniques such as heat sealing, solvent bonding, adhesive bonding or use of coatings. As will be described in further detail hereinbelow, in a preferred embodiment of the present invention, the one or more laminates of polymeric material, preferably supplied in sheets or tubes, are positioned about the intermediate member. Pressure is used to compress against one surface of the intermediate member, forcing the laminate to conform to the open configuration of the intermediate member. Once so conformed, fusion is effected between the layers so as to form the reservoir pocket, with the fusion occurring within the open spaces defined by the open construction. In one embodiment, this fusion may occur at a location substantially coextensive with the inner surface of the intermediate member.

In one preferred embodiment, the intermediate structure member is a stent having an open construction, such as that described in U.S. Pat. No. 6,139,573, where the polymeric liners are adhered together through the openings of the stent. It is also well within the contemplation of the present invention that the intermediate structure member can be defined by foreign bodies and openings between the foreign bodies. As defined herein, “foreign bodies” includes particles, fibers, wires and inclusions. Foreign bodies can be metallic, polymeric, mineral, ceramic, salts, or other materials which serve to produce pockets in the completed laminated or bonded structure. The foreign bodies can comprise dissolvable or biodegradable material. In one embodiment, the device of the present invention is a vascular graft which does not require a stent component, but may use fibers or wires to provide strength or kink resistance, or may utilize particles or inclusions simply for the function of producing pockets in the completed structure. Another embodiment includes an apposition means which hold the device against a vessel wall, but do not necessarily provide significant stenting force on the vessel. Yet another embodiment includes stent components at certain locations, such as at each end and/or isolated regions along the length of the device. Still another embodiment includes stent components at one or more regions, and includes other wires, fibers, particles, or inclusions to provide additional pockets; these additional components can be located along the entire length of the device, or at selected regions such as those regions which do not have a stent component, and can provide additional function as well, such as strengthening, kink resistance, or radiological contrast or other imaging enhancement.

The present invention is particularly suitable for forming an endoluminal prosthesis for vascular applications. For example, an expandable stent is encased in multiple layers of a polymeric material, preferably expanded polytetrafluoroethylene (ePTFE). The ePTFE layers are fused together through the open construction of the stent so that the covered stent exhibits a relatively smooth surface as compared with an uncovered stent. This type of smooth stent has the tendency to reduce thrombotic formation following vascular implantation and to impart less turbulence to the fluid flowing therethrough.

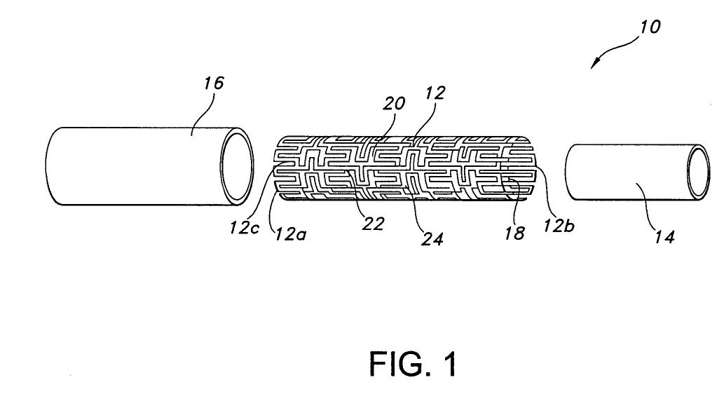

Referring now to the drawings of the present invention, FIG. 1 shows a composite tubular endoprosthesis 10, which is formed by combining an open construction stent 12 between an inner tubular liner 14 and an outer tubular liner 16. Stent 12 is generally an elongate tube having opposed ends 12a and 12b, and a central lumen 12c therebetween. The body of stent 12 defines an interior surface 18 and an opposed exterior surface 20. The stent is formed to have a generally open configuration having a plurality of spaces 22 and a solid portion 24 of the body. These openings 22 provide the longitudinal flexibility of the stent, as well as to permit the stent to be radially expanded once deployed in a body lumen, such as a blood vessel.

Polymeric liners 14 and 16 of the present invention may be formed by a variety of methods. For example, extrusion processes such as ram extrusion; polymeric casting techniques such as solvent casting and film casting; molding techniques such as blow molding, injection molding and rotational molding; and other thermo-forming techniques useful with polymeric materials may be employed and chosen to best serve the type of material used and specific characteristics of the liner desired.

Moreover, while either or both of the polymeric liners may be provided directly in tubular form, i.e. as an extruded tube, either one or both can also be formed from extruded sheets of material which can be wrapped around the stent to form a liner. Combinations of sheets and tubes are also contemplated.

The support structure of the composite device of the present invention may be chosen from a wide variety of materials and configurations. Endovascular stents are the preferred support structure and may be formed in a wide variety of configurations. An example of a useful stent in the present invention is shown in FIG. 1. This particular stent represents a slotted tubular stent which is designed to radially expand either by balloon catheter or by forming the stent from a temperature-sensitive memory alloy which changes shape at a designated temperature or temperature range. Other stent types, such as tubular-shaped wire stents and self-expandable spring-biased stents are also contemplated. The stent may be made from a variety of materials including stainless steel, titanium, platinum, gold, tantalum and other biocompatible metals. In addition, thermoplastic materials which are inert in the body may also be employed. Shaped memory alloys having super elastic properties generally made from specific ratios of nickel and titanium, commonly known as nitinol, are among the preferred stent materials.

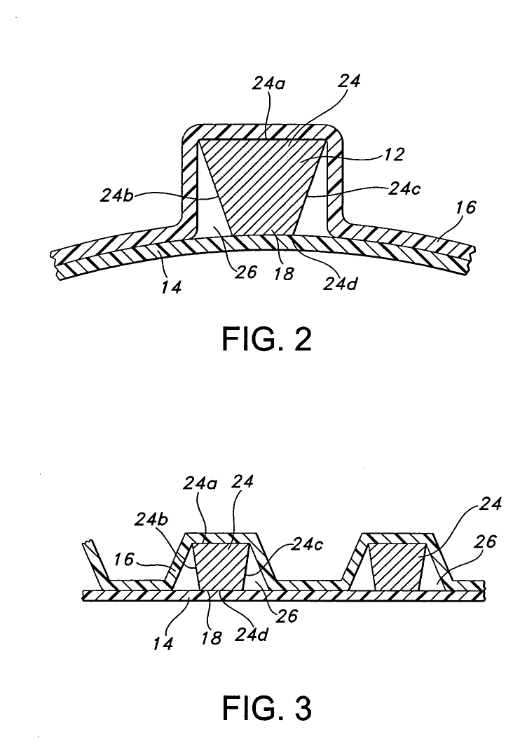

Referring now to FIGS. 2 and 3, which show the device prior to incorporation of the bioactive agent, inner tubular liner 14 and outer tubular liner 16 are shown encasing the solid portions 24 of stent 12. Liners 14 and 16 substantially cover the solid portion 24 of stent 12. This results in the outer tubular liner 16 covering an upper surface portion 24a of solid portion 24, as well as a substantial extent of depending opposed side surface portions 24b and 24c thereof. Opposed lower surface portion 24d of the solid portion 24 is covered by inner tubular liner 14. It is only necessary to enclose or envelope surface portions 24a-24d of stent 12 with liners 14 and 16. In the embodiments shown in FIGS. 2 and 3, upper and lower surface portions 24a, 24b are covered by liners 16 and 14, respectively, and opposed side portions 24b and 24c are enclosed thereby. In one embodiment, liner 16 is conformed to at least a portion of side segment surfaces 24b and 24c.

As shown in FIGS. 2 and 3, inner tubular liner 14 and outer tubular liner 16 are joined to form a reservoir pocket 26 about solid segments 24 for containing various bioeffecting agents therewithin. Pockets 26 formed by the joining of liners 14 and 16 are adjacent to the stent segments 24. The embodiments shown in FIGS. 2 and 3 show the joining of liners 14 and 16 occurring at a location substantially coextensive with interior surface 18 of stent 12, this interior surface being defined by inner segment surface 24d. It is noted, however, that it is well within the contemplation of the present invention that the location at which liner 14 and liner 16 are joined may be at a location which is not coextensive with the interior surface of the stent.

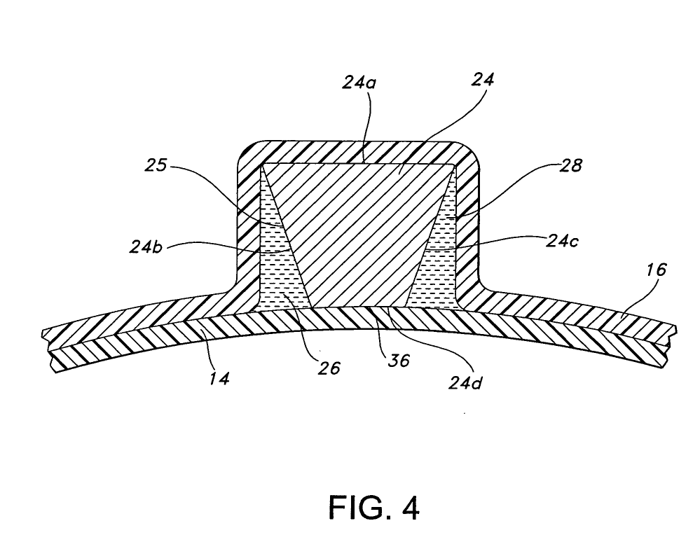

Referring now to FIG. 4, the device of the present invention is shown following incorporation of a fluid mixture containing a bioactive agent 28 within the reservoir pocket 26. Similarly, a gel containing the bioactive agent may be incorporated within the reservoir pocket.

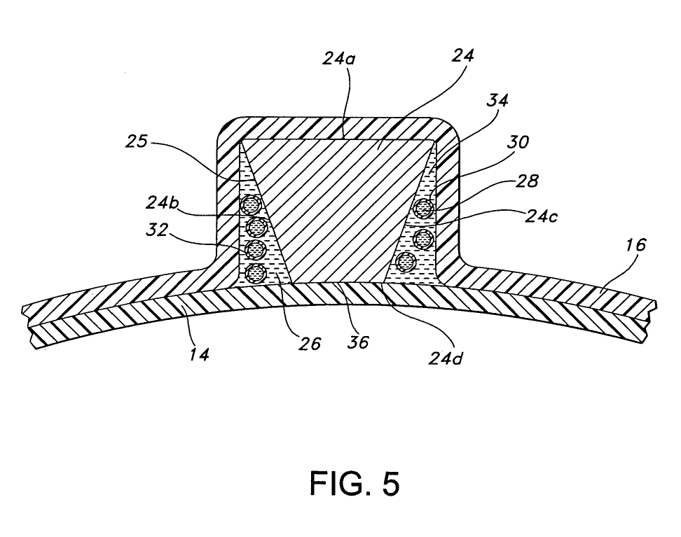

FIGS. 5 illustrates an embodiment wherein a bioactive agent 28 is first incorporated within a polymeric shell 30 to form a drug-containing microparticle 32 (e.g. microsphere), which can then be mixed with fluid or gel 34 for incorporation within reservoir pocket 26. As will be described in further detail below, it is further contemplated that a bioactive agent may be incorporated within a non-hollow microparticle, which may be loaded within the reservoir pocket. Such embodiments will be described in further detail below.

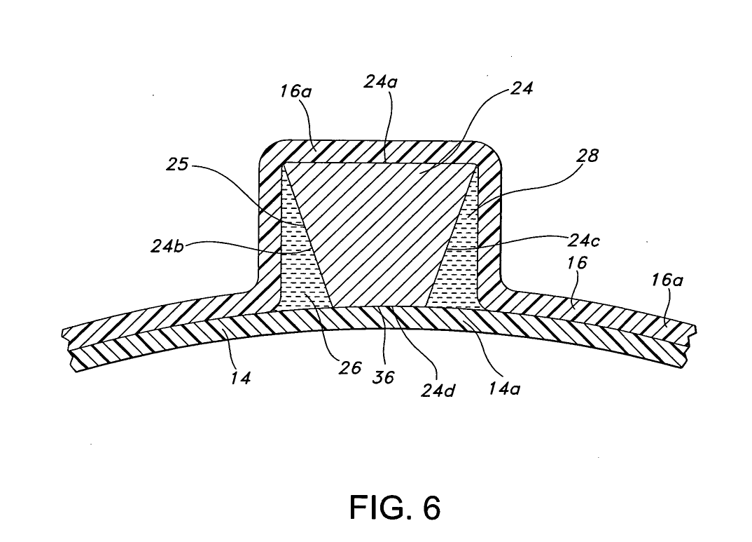

With reference now to FIG. 6, it is a further aspect of the device of this invention that at least one of the liners is porous so as to allow the bioactive agent to be delivered to the site of implantation of the device. In embodiments where both liners are porous, the porosities of the first and second polymeric liners may be designed to be different. For example, as shown in FIG. 6, first polymeric liner 14 has a porosity 14a which is different from the porosity 16a of liner 16. In particular, luminal liner 14 may be chosen to exhibit a radial strength in excess of the radial strength of the second liner 16. Alternatively, the porosities of the first and second polymeric liners may be designed such that the second liner 16 exhibits a radial strength in excess of the radial strength of the inner (i.e. luminal) liner 14. Furthermore, the porosities of the first and second polymeric liners may be designed so as to achieve a certain structure and geometry of the nodes and fibrils that affect permeability and prevent tissue in-growth. This will be described in further detail below.

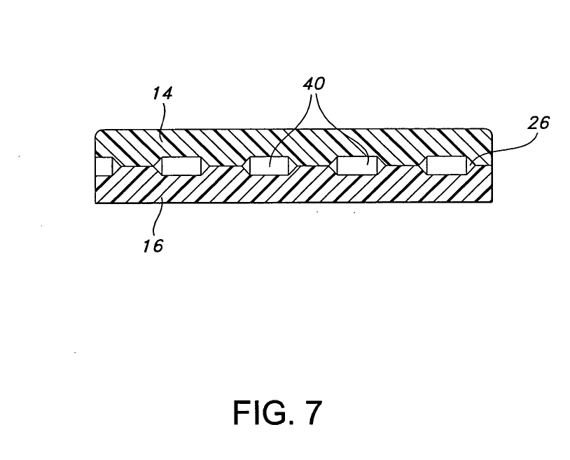

Referring now to FIG. 7, a vascular graft of the present invention is shown in which interposed between an inner polymeric liner 14 and an outer polymeric liner 16 are foreign bodies 40, such as inclusions or reinforcing fibers. The intermediate foreign bodies 40 form pockets 26 between the inner and outer liners for containing a bioactive agent. As described above, the foreign bodies can be particles, wires, fibers or inclusions. Fibers or wires can provide strength or kink resistance to the composite device, or particles or inclusions can be included simply for the function of producing the pockets in the completed device.

One object of the present invention is to provide an implantable composite device for delivery of bioactive agents associated therewith to a site of implantation of the device. For drug delivery, it is recognized that it is difficult to obtain constant drug delivery when administering the drug in the form of pills and injections. As a result of repeated doses, the drugs often cycle through concentration peaks and valleys, thus resulting in time periods of toxicity and ineffectiveness. Thus, localized drug delivery is desired.

The composite device of the present invention includes at least one bioactive agent which will be released from the device at the implantation site in order to supply the bioactive agent where it is needed without the problems associated with systemic delivery. The rate at which the bioactive agent is released from the inventive device depends on the size and the number of pores in the reservoir pocket's walls and the size of the bioactive agent molecule. The pore size of the liners can be selected so that the bioactive agent remains in the pockets for a desired period of time. A desired release rate can be chosen for a particular bioactive agent, for example, depending on its size, stability, potency, etc. The dimensions of the pockets can also be selected to achieve a desired release rate for the bioactive agent. In addition, the pocket dimensions can be selected to contain a desired quantity of a bioactive agent. The pocket size can be large compared to the pore size of one or more of the liners.

The composite device of the present invention may achieve localized delivery of a bioactive agent to a site where it is needed in a number of ways. In one embodiment, the reservoir pocket is directly filled with a fluid or gel containing the bioactive agent. In another embodiment, the bioactive agent is first encapsulated in a polymer, i.e. matrix. One example of a suitable polymeric matrix for encapsulating purposes is polystyrene-polyisobutylene-polystyrene (SIBS). The polymeric matrix containing the bioactive agent may include, without limitation, microspheres, microsponges, microfibers or microfibrils, which are loaded into the reservoir pocket. These may be hollow or non-hollow. Suitable microparticles are described in U.S. Pat. No. 5,290,271 to Jernberg. In the case of microsponges, the bioactive agent is contained within their microchanneling. In one embodiment, these microparticles are mixed with a fluid or gel and injected into the reservoir pocket, or delivered to the reservoir pocket by way of a pump or mini-pump attached to the reservoir pocket. The fluid or gel mixed with the microparticles could be a carrier agent designed to improve the cellular uptake of the bioactive agent incorporated into the composite device. An example of a carrier agent would be hyaluronic acid, or its derivatives, which may be incorporated within the fluid or gel.

The microparticles in the reservoir pocket may have a polymeric shell surrounding the bioactive agent. Alternatively, the bioactive agent may be embedded within a non-hollow microparticle. Due to the potential for varying thicknesses of the polymeric matrix and for varying porosities and permeabilities of different polymeric materials suitable for containing a bioactive agent, there is provided the potential for a mechanism for controlling the release of a therapeutic agent in a highly regulated manner. For example, pore size and pore number for the polymeric matrix can be selected to achieve a desired release rate for a particular bioactive agent, depending on its size, potency, or stability.

Various methods are known for encapsulating drugs, within microparticles or microfibers. For example, see Patrick B. Deasy, Microencapsulation and Related Drug Processes, Marcel Dekker, Inc., New York, 1984, which provides example methods used to prepare microspheres which incorporate bioactive agents and optimal carrier agents. Moreover, hollow microfibers in the range of size of 100 to 1,000 microns in diameter can be produced and drug loaded by extrusion.

With reference to FIGS. 4-6, wall 36 of reservoir pocket 26 may be formed of a porous polymeric material which is sufficiently permeable to permit a bioactive agent disposed in the reservoir pocket to diffuse through wall 36 and to a site of implantation. In addition, in cases where the solid stent segments 24 are partly or wholly formed from a biostable polymer, the polymer may be sufficiently permeable to permit diffusion of the bioactive agent through wall 25 and subsequently wall 36 to the site where it is needed. Depending on the nature and porosity of the material used to form the inner liner or solid stent segments, regulation of the flow and release of a bioactive agent to the implantation site may be possible.

The bioactive agents which achieve regulated and specific delivery through their association with the composite device of the present invention, may be selected from silver antimicrobial agents, metallic antimicrobial materials, growth factors, anti-coagulant substances, stenosis inhibitors, thrombo-resistant agents, antibiotic agents, anti-tumor agents, anti-proliferative agents, growth hormones, antiviral agents, anti-angiogenic agents, angiogenic agents, anti-mitotic agents, anti-inflammatory agents, cell cycle regulating agents, genetic agents, cholesterol-lowering agents, vasodilating agents, agents that interfere with endogenous vasoactive mechanisms, hormones, their homologs, derivatives, fragments, pharmaceutical salts and combinations thereof.

Suitable bioactive agents can also include diagnostic agents or media loaded into the pockets, such as including radiologic contrast materials, radiopaque materials, MRI contrast agents, ultrasound contrast agents, or other imaging aids such as iodinated or non-iodinated contrast media, metallic materials such as gold, iridium, platinum, palladium, barium compounds, gadolinium, encapsulated gas, or silica.

Cells can be associated with the composite device of the present invention. For example, cells that have been genetically engineered to deliver bioactive proteins, such as the above-mentioned growth factors or antibodies, to the implant site can be associated with the composite device of the present invention. Cells can be of human origin (autologous or allogeneic) or from an animal source (xenogeneic). Cells can be pre-treated with medication or pre-processed such as by sorting or encapsulation. The delivery media can be formulated as needed to maintain cell function and viability. A suitable means of delivery of genetically-engineered cells to the implantation site may be by use of the pocket reservoir of the inventive composite device.

Thrombo-resistant agents associated with the composite device may be selected from the following agents: heparin, heparin sulfate, hirudin, hyaluronic acid, chondroitin sulfate, dermatan sulfate, keratan sulfate, PPack (dextrophenylalanine proline arginine chloromethylketone), lytic agents, including urokinase and streptokinase their homologs, analogs, fragments, derivatives and pharmaceutical salts thereof.

Anti-coagulants may be selected from the following: D-Phe-Pro-Arg chloromethyl keton, an RGD peptide-containing compound, heparin, antithrombin compounds, platelet receptor antagonists, anti-thrombin antibodies, anti-platelet receptor antibodies, aspirin, prostaglandin inhibitors, platelet inhibitors, tick antiplatelet peptides and combinations thereof.

Suitable antibiotic agents can include, but are not limited to, the following agents: penicillins, cephalosporins, vancomycins, aminoglycosides, quinolones, polymyxins, erythromycins, tetracyclines, chloramphenicols, clindamycins, lincomycins, sulfonamides their homologs, analogs, derivatives, pharmaceutical salts and combinations thereof.

Anti-proliferative agents for use in the present invention include, but are not limited to the following: enoxaprin, angiopeptin, or monoclonal antibodies capable of blocking smooth muscle cell proliferation, hirudin, acetylsalicylic acid, and combinations thereof.

Useful vascular cell growth inhibitors include, but are not limited to, the following: growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin.

Suitable vascular cell growth promoters include, but are not limited to, transcriptional activators and transcriptional promoters.

Useful anti-tumor agents for use in the present invention include, but are not limited to, paclitaxel, docetaxel, alkylating agents including mechlorethamine, chlorambucil, cyclophosphamide, melphalan and ifosfamide, antimetabolites including methotrexate, 6-mercaptopurine, 5-fluorouracil and cytarabine, plant alkaloids including vinblastine, vincristine and etoposide, antibiotics including doxorubicin, daunomycin, bleomycin, and mitomycin, nitrosureas including carmustine and lomustine, inorganic ions including cisplatin, biological response modifiers including interferon, angiostatin agents and endostatin agents, enzymes including asparaginase, and hormones including tamoxifen and flutamide their homologs, analogs, fragments, derivatives, pharmaceutical salts and combinations thereof.

Furthermore, anti-viral agents include, but are not limited to, amantadines, rimantadines, ribavirins, idoxuridines, vidarabines, trifluridines, acyclovirs, ganciclovirs, zidovudines, foscarnets, interferons their homologs, analogs, fragments, derivatives, pharmaceutical salts and mixtures thereof.

Useful anti-inflammatory agents include agents such as: dexamethasone, prednisolone, corticosterone, budesonide, estrogen, sulfasalazine, mesalamine, and combinations thereof.

In one desired embodiment, an anti-mitotic agent may be a radioactive material coupled to a biologically compatible carrier. In particular, the radioactive material may be selected from alpha-particle emitting isotopes and beta-particle emitting isotopes. Useful beta-particle emitting isotopes for treatment are generally selected from ³²P, ¹³¹I, ⁹⁰Y and mixtures thereof.

In other embodiments, the bioactive agent associated with the composite device of the present invention may be a genetic agent. Examples of genetic agents include DNA, anti-sense DNA, and anti-sense RNA. DNA encoding one of the following may be particularly useful in association with an implantable device according to the present invention: (a) tRNA or rRNA to replace defective or deficient endogenous molecules; (b) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor; (c) cell cycle inhibitors; (d) thymidine kinase and other agents useful for interfering with cell proliferation; and (e) the family of bone morphogenic proteins. Moreover DNA encoding for molecules capable of inducing an upstream or downstream effect of a bone morphogenic protein may be useful.

The first and second liners may be formed from a synthetic polymer, natural polymer, or a combination thereof. Synthetic polymers may include, but are not limited to, fluoropolymers, polyurethanes, polyurethane ethers, polyurethane esters, polyurethaneureas, polyacrylamides, polyvinyl alcohols, polyphosphate esters, polyethersulfone, polyorthoesters, polyesters, siloxane polymers, silicones, polyvinylpyrrolidone, polyvinyl ethers, polyethers, polycarbonate, polyalkylenes, polyamides, polyanhydrides, polyethylene oxides, polyvinyl aromatics, polyhydroxybutyrate valerate, polyhydroxybutyrate-co-hydroxyvalerate, polyacrylic acid, polyhydroxybutyrate valerate, polyhydroxybutyrate-co-hydroxyvalerate, polyacrylic acid and derivatives and mixtures thereof. In one preferred embodiment, the synthetic polymer is ePTFE.

Moreover, the natural polymer forming the polymeric liners is desired to be selected from the following: fibrin, elastin, celluloses, collagen, gelatin, vitronectin, fibronectin, laminin, reconstituted basement membrane matrices, starches, dextrans, alginates, hyaluronic acid, polylactic acid, polyglycolic acid, polypeptides, glycosaminoglycans, their derivatives and mixtures thereof.

In one embodiment, the natural and synthetic polymers forming the first and second polymeric liners are biostable or bioabsorbable polymers. Moreover, the stent segments may be partly or wholly formed from a biostable or bioabsorbable polymer. Wherein the composite device is biostable, the bioactive agent may diffuse out from the reservoir pocket to the site where it is needed. If, however, the inner polymeric liner or solid stent segments are bioabsorbable, a bioactive agent incorporated within the reservoir pocket may be delivered to a site where it is needed in part by the process of degradation and resorbtion of the polymer itself.

While biological polymeric materials such as fibrin, collagen, and elastin possess high biocompatibility per se, their mechanical properties are often inadequate and, furthermore, they cost much more to produce than synthetic polymers. Therefore, synthetic and biological polymers may be combined in order to produce a device having the superior mechanical properties associated with a synthetic component and the biocompatibility associated with a biological component. Moreover, the blending of synthetic and biological polymers offers increased flexibility in terms of the porosity and permeability of the resultant combined blend for increased ability to affect in a specific way the size of the bioactive agent capable of diffusing through the layer and the delivery rate thereof to the site of implantation. Blending techniques are well known such as those described in the International Journal of Artificial Organs, Vol. 14, No. 5, 1991, pp 295-303.

The porosity of the first and second polymeric liners (14 and 16, respectively) may be designed to achieve desirable properties in the structure and geometry of the nodes and fibrils that affect permeability and prevent tissue in-growth. The inner liner 14, which forms one of the walls (wall 36) of the reservoir pocket 26, may have a specific node/fibril geometry and sufficient fibril density to allow regulated delivery of the bioactive agent to the implantation site. In one desired embodiment, the inner liner 14 is a layer formed of ePTFE having pores of an internodal distance from about 5 to about 10 microns. In a further embodiment, the inner liner of ePTFE has a specific node/fibril geometry of about 5 to about 10 microns.

It is a further aspect of the devices of this invention that the porosities of the first and second polymeric liners may be designed to increase radial and suture retention strengths of the composite device, as well as promote enhanced cell endothelization, preferably along the inner luminal surface of the graft. In one desired embodiment, the second liner 16 exhibits a radial strength in excess of the radial strength of the inner (i.e. luminal) liner 14. For example, in this case, the second liner 16 has pores of an internodal distance of less than 40 microns, whereas the luminal zone 14 of ePTFE has pores of an internodal distance of greater than 40 microns. The larger IND associated with the luminal layer 14 is designed to enhance cell endothelization along the luminal surface as the graft is inherently more porous and this contributes to long term healing and patency of the graft. The decrease in the porosity of the second liner 16, relative to luminal liner 14, results in an increase in the overall radial tensile strength of the device, as well as an increase in the ability for the graft to retain a suture placed therein during implantation.

It is an additional object of the present invention to provide a composite device wherein the luminal liner 14 exhibits a radial strength in excess of the radial strength of the second liner 16. In this instance, the second liner provides a porosity sufficient to promote enhanced cell growth and tissue incorporation, hence more rapid healing, and the inner luminal liner has a high degree of strength. In one embodiment, the first luminal liner 14 of ePTFE has pores of an internodal distance of less than 40 microns and the second liner 16 of ePTFE has pores of an internodal distance of greater than 40 microns.

Useful ranges of internodal distance for ePTFE materials include the range of approximately 20 to 120 micrometers average internodal distance, although the invention is not limited to this range, as described above.

The composite device according to the present invention may be formed by adheringly supporting tubular structures over one another to form a composite tubular graft as described in further detail below. Moreover, the method may further include interposing an implantable prosthetic stent between the layers and incorporating a bioactive agent into the device.

The stent 12 used in the stent-graft arrangement may be of any stent configuration known to those skilled in the art. Various stent types and stent constructions may be employed in the present invention including, without limitation, self-expanding stents and balloon expandable stents. The stents may be capable of radially contracting as well. Self-expanding stents include those that have a spring-like action which cause the stent to radially expand or stents which expand due to the memory properties of the stent material for a particular configuration at a certain temperature. Other materials are of course contemplated, such as stainless steel, platinum, gold, titanium, tantalum, niobium, and other biocompatible materials, as well as polymeric stents. The configuration of the stent may also be chosen from a host of geometries. For example, wire stents can be fastened in a continuous helical pattern, with or without wave-like forms or zigzags in the wire, to form a radially deformable stent. Individual rings or circular members can be linked together such as by struts, sutures, or interlacing or locking of the rings to form a tubular stent.

A suitable method of forming a composite endoluminal device of the present invention includes the steps of providing the inner liner on an elongate mandrel. An elongate expandable stent having a cylindrical body and defining an interior surface, and exterior surface, and having openings therethrough is then positioned over the inner liner and engaged thereto. Subsequently, an outer stent liner is positioned over the stent to form a stent assembly including the inner liner, stent, and outer liner. The outer liner is preferably compressed through the openings of the stent and into contact with the inner liner. The outer liner is then adhered or otherwise laminated or bonded to the inner liner so as to form the reservoir pocket, which is subsequently filled with a bioactive agent so as to yield the inventive device. It is noted that the present invention also contemplates heating the stent assembly while it is still on the mandrel to heat shrink the outer liner 16 and inner liner 14 about the stent. A suitable method for forming the stent assembly prior to incorporation of the bioactive agent is provided in U.S. Pat. No. 6,139,573, the entire contents of which are herein incorporated by reference.

With reference to the methods and conditions under which the first 14 and second 16 polymeric liners are formed, as with the known methods of processing PTFE, the method for preparing the liners of ePTFE utilizes a preformed billet which includes a PTFE resin mixed with an organic solvent. It is noted that extrusion conditions have a large effect on an extrudate's reaction to being stretched. For example, extrudate qualities may be controlled by a number of factors including the amount of organic solvent mixed with the resin to form a billet, the reduction ratio at which the billet is extruded and the extrusion rate. Each of these is believed to influence the micromechanical properties of the extruded article. U.S. Pat. No. 5,433,909 provides a method for forming controlled porosity ePTFE products.

A billet which has a solvent level of about 10 to 30% by weight yields an extrudate suitable for the stretching process necessary to produce a first luminal liner 14 capable of regulating delivery of bioactive agents from the reservoir pocket to the implantation site. Moreover, it is desired that the preformed billet is extruded to a reduction ratio of about 200 to 1. An additional parameter which has a significant effect on the resulting extrudate property upon being stretched is the extrusion pressure. Suitable extrusion pressures to practice the present invention include pressures of about 5,000 PSI to about 10,000 PSI.

As mentioned above, in one embodiment, the location at which the inner 14 and outer 16 liners are joined is a location substantially coextensive with the interior surface 18 of the stent. It is contemplated by the present invention that the coextensive location includes an area slightly interior of the interior surface where the outer liner is compressed fully through the openings of the stent. It is also contemplated that the location coextensive with the interior surface of the stent also includes an area slightly exterior of the interior surface within the stent openings where the stent is self-compressed upon the inner liner so that uncompressed portions of the liner may break the plane of the interior stent surface by extending into the openings.

The method of forming the composite intraluminal device of the present invention may include laminating, adhering, or bonding the outer liner to the inner liner in a manner such that the outer liner substantially conforms to the complex geometry provided by the exterior surface and the openings of the stent. This enables the device to exhibit substantial benefits in endovascular use as the stent is substantially covered with a biocompatible thrombus-inhibiting material which encourages tissue in-growth and maintains metabolic communication across the outer liner 16 and inner liner 14. In addition, as the inner luminal surface of the endoprosthesis is formed against a smooth mandrel in certain embodiments, the inner luminal surface of the composite endoprosthesis exhibits a relatively smooth configuration mitigating against turbulent blood flow and thrombis formation during use.

Desirably, the step of compressing the outer liner 16 to contact the inner liner 14 is performed by forcing a flowable mass against the outer liner 16, so as to force-outer liner to the openings 22 of the stent. The flowable mass is desirably formed from a flowable particulate such as granules or grains of salt, and/or other material capable of transmitting a compaction force fluidly and substantially uniformly to the contour of the stent. This flowable particulate is desirably capable of withstanding temperatures which permit the confirmation of the outer liner about and through the openings of the stent and fuse the outer liner 16 to the inner liner 14 therethrough. Most desirably, the particulate flowable mass is water soluble to facilitate removal of particles during washing steps in the manufacturing and assembly process. In one embodiment, the particulate flowable mass is formed by a composition including sodium chloride. The composition may also include an anti-caking agent or flow acid, such as tricalciumphosphate and the like.

In order to function effectively as a drug- or bioactive agent-delivering endoprosthesis, the reservoir pocket must be formed through the joining of the inner and outer tubular liners. These liners may be laminated together through the open construction of the stent so as to form an ePTFE covered composite endoprosthesis, in preferred embodiments. As mentioned above, numerous techniques may be employed to laminate or bond the inner tubular liner to the outer tubular liner through the open spaces of the stent. For example, heat setting, adhesive welding, application of uniform force, and other bonding techniques may all be employed to bond or secure the inner liner to the outer liner through the stent. Whereas it is contemplated that liners 14 and 16 may be adhered at a location substantially coextensive with the interior surface 18 of the stent, it is further contemplated that the joining may occur at other locations. In those instances where the adherence occurs at a location coextensive with the interior surface 18 of the stent, this is especially beneficial for maintaining the smoothness of the inner luminal surface, so as to minimize the turbulence of or the interference with the fluid flowing through the device while also minimizing the risk of thrombis formation.

It is within the contemplation of the present invention that the bioactive agent is incorporated within the composite structure of this invention either prior to, during, or following implantation. For example, a bioactive agent contained within the reservoir pocket may be incorporated within the structure of the device during the method of making and, following implantation, the drug can be delivered to the reservoir pocket by use of a mini-pump which can be attached to the reservoir of the device, for example, by a catheter. The mini-pump may be located at a site external to the patient or may be surgically implanted. Alternatively, a bioactive agent may be added to the reservoir pocket prior to implantation of the device, such as by pre-filling the reservoir pocket with a syringe.

In one embodiment, a reservoir pocket is filled with bioactive agent by applying a vacuum. For example, at least a portion of the device can be confined, and a vacuum is pulled in the confined space to evacuate the pockets. Subsequently, the bioactive agent is introduced in the confined space so that it will be drawn into the pockets.

In a preferred embodiment, the reservoir pocket is filled with a bioactive agent by applying a vacuum and pressure to the device after the reservoir pocket is formed in order to remove air from the pocket for replacement with a bioactive agent fluid in which the device under vacuum and pressure has been immersed. For example, preferably at least a portion of the device is first confined, and a vacuum is pulled in the confined space to evacuate the pockets. The bioactive agent is then introduced in the confined space so that it will be drawn into the pockets and pressure is then applied in the confined space to force additional agent into the pockets.

In yet another embodiment, the pockets are first pre-treated with supplementary material, such as a surfactant or other chemicals to aid in incorporating the bioactive agent into the pockets or to help the agent retain activity or viability. For example, the pocket can be filled with a surfactant solution by applying a vacuum and pressure to the device to evacuate air from the pockets for replacement with the surfactant. A vacuum can then be pulled to remove some of the surfactant solution. The bioactive agent is next introduced into the pockets under vacuum and pressure.

In any of these embodiments for filling the reservoir pocket(s), excess bioactive agent may be flushed or rinsed out of any large pores in the ePTFE, leaving the bioactive material remaining in the reservoir pocket, which desirably modulates the release of the agent to the implantation site. The bioactive agent can be in the form of a liquid, gas, vapor, suspension, etc. to facilitate loading in the pockets.

Furthermore, a bioactive agent or drug can be incorporated into the polymeric material of a microparticle, such as a microsphere, microfiber, or microfibril in the following manner: mixing into an extrudate used to make the polymeric matrix of the microparticle, a crystalline, particulate material like salt or sugar that is not soluble in a solvent used to form the extrudate; casting the extrudate solution with particulate material; and then applying a second solvent, such as water, to dissolve and remove the particulate material, thereby leaving a porous polymeric matrix. The porous matrix may then be placed into a solution containing a bioactive agent in order to fill the pores. Preferably, a vacuum would be pulled on the porous matrix to insure that the bioactive agent applied to it is received into the pores. The encapsulated bioactive agent can then be mixed with a fluid or gel for delivery thereof to the pockets.

As described above, the present invention is further directed toward treating a lumen in a body by: inserting a generally cylindrical implantable composite device for delivery of bioactive agents incorporated therewith into the lumen, the device including a first polymeric liner; a second polymeric liner; an intermediate structural member interposed between the liners, the intermediate structural member being defined by solid segments and openings therebetween such that the liners can be bonded together through the openings to form at least one pocket about the solid segments; and a bioactive agent located within the pocket(s). The method also includes affixing the implantable composite device to the lumen such that it will stay where positioned.

In one specific embodiment, the device for implantation is a stent-graft composite device, the stent having a generally cylindrical tubular body defined by the solid segments and openings between the solid segments. In other embodiments, the device for treating a lumen in the body is a vascular graft with inclusions or reinforcing fibers, a covered bioresorbable stent, or other similar tubular device formed of multiple layers to create pockets adjacent to at least one of the solid segments of the intermediate structure member interposed between the layers. Implantation can be by open or minimally invasive surgical access, or can be by percutaneous access, such as with catheters and the like.

As described above, the pockets can be loaded with bioactive material at the time of implant, or can be loaded at some time prior to implant. Loading the pockets with bioactive material can be performed by applying suction to evacuate the pockets. Subsequently, the bioactive agent is introduced into the pockets. Additional bioactive material can be incorporated by applying pressure to force the additional material into the pockets.

Alternatively, a previously implanted device can be accessed for reloading the pockets with bioactive material. The step of accessing the device can be performed by open or minimal invasive surgical access, or can be by percutaneous access such as with catheters and the like. As described above, the pockets can be loaded by providing containment means, and applying suction (to remove unwanted material from the pockets) or pressure (to force material into the pockets) or suction (to remove unwanted material from the pockets) followed by pressure (to force material into the pockets). The containment means can include at least one balloon on a catheter device, or an isolation member (such as a membrane) positioned in apposition to the implantable device, or can contain an external sheath applied by surgical access, or a combination of these or other containment components.

1. A composite device for delivery of bioactive agents associated therewith to a site of implantation of said device comprising:
a first polymeric liner;
a second polymeric liner;
an intermediate structural member interposed between said first and said second polymeric liners, said intermediate structural member being defined by solid segments and openings therebetween such that the first liner can be bonded to the second liner through said openings to form at least one pocket about said solid segments; and
a bioactive agent located within said pocket about said solid segments of said intermediate structural member.

2. The device of claim 1, wherein said intermediate structure member is a stent having a generally cylindrical tubular body defined by said solid segments and said openings therebetween, said tubular body defining an inner surface and an opposed outer surface.

3. The device of claim 2, wherein said first and said second liners are adheringly joined at a location substantially coextensive with said inner surface of said tubular body.

4. The device of claim 2, wherein said solid stent segments include opposed inner and outer segment surfaces defining said inner and outer surfaces of said tubular body and opposed side segment surfaces between said inner and outer segment surfaces.

5. The device of claim 4, wherein said second liner is conformed to at least a portion of said side segment surfaces.

6. The device of claim 2, wherein said first polymeric liner is positioned about said inner surface of said tubular body.

7. The device of claim 2, wherein said second polymeric liner is positioned about said outer surface of said tubular body.

8. The device of claim 1, wherein said first liner defines a fluid contacting luminal surface.

9. The device of claim 1, wherein said bioactive agents in said reservoir are selected from the group consisting of antimicrobial agents, growth factors, anti-coagulant substances, stenosis inhibitors, thrombo-resistant agents, antibiotic agents, anti-tumor agents, anti-proliferative agents, growth hormones, antiviral agents, anti-angiogenic agents, angiogenic agents, anti-mitotic agents, anti-inflammatory agents, cell cycle regulating agents, genetic agents, cholesterol-lowering agents, vasodilating agents, agents that interfere with endogenous vasoactive mechanisms, hormones, their homologs, derivatives, fragments, pharmaceutical salts and combinations thereof.

10. The device of claim 1, wherein said solid segments of said intermediate structural member are foreign bodies, forming said pockets between said first and second liners thereabout.

11. The device of claim 1, wherein said bioactive agents in said reservoir are encapsulated in a polymeric matrix.

12. The device of claim 11, wherein said polymeric matrix containing said bioactive agent is a microparticle, microfiber or microfibril.

13. The device of claim 1, wherein said first liner and said second liner are independently selected from the group consisting of synthetic polymer, natural polymer or a combination thereof.

14. The device of claim 1, wherein at least one of said first or said second liners is porous.

15. The device of claim 13, wherein said synthetic polymer is selected from the group consisting of fluoropolymers, polyurethanes, polyurethane ethers, polyurethane esters, polyurethaneureas, polyimides, polyacrylamides, polyvinyl alcohols, polyphosphate esters, polyethersulfone, polyorthoesters, polyesters, siloxane polymers, silicones, polyvinylpyrrolidone, polyvinyl ethers, polyethers, polycarbonate, polyalkylenes, polyamides, polyanhydrides, polyethylene oxides, polyvinyl aromatics, polyhydroxybutyrate valerate, polyhydroxybutyrate-co-hydroxyvalerate, polyacrylic acid and derivatives and mixtures thereof.

16. The device of claim 13, wherein said synthetic polymer is ePTFE.

17. The device of claim 13, wherein said natural polymer is selected from the group consisting of fibrin, elastin, celluloses, collagen, gelatin, vitronectin, fibronectin, laminin, reconstituted basement membrane matrices, starches, dextrans, alginates, hyaluronic acid, polylactic acid, polyglycolic acid, polypeptides, glycosaminoglycans, their derivatives synthetic analogs and mixtures thereof.

18. The device of claim 13, wherein said natural polymer and said synthetic polymer are biostable or bioabsorbable polymers.

19. The device of claim 2, wherein said stent is a biocompatible metal.

20. The device of claim 19, wherein said biocompatible metal is selected from the group consisting of stainless steel, platinum, gold, nitinol, tantalum and alloys thereof.

21. The device of claim 1, wherein said first and said second liners are of ePTFE.

22. The device of claim 14, wherein the porosity of said first liner is different from the porosity of said second liner.

23. The device of claim 21, wherein said first liner of ePTFE has pores of an internodal distance of greater than 40 microns and said second liner of ePTFE has pores of an internodal distance of less than 40 microns.

24. The device of claim 23, wherein said second liner exhibits a radial strength in excess of the radial strength of said first liner.

25. The device of claim 21, wherein said first liner of ePTFE has pores of an internodal distance of less than 40 microns and said second liner of ePTFE has pores of an internodal distance of greater than 40 microns

26. The device of claim 25, wherein said first liner exhibits a radial strength in excess of the radial strength of said second liner.

27. A composite intraluminal device for delivery of bioactive agents associated therewith to a site of implantation of said device comprising:
an elongate stent having a generally cylindrical tubular body defined by solid segments and openings between said solid segments, said tubular body defining an inner surface and an opposed outer surface;
a first polymeric liner positioned about said inner surface of said tubular body;
a second polymeric liner positioned about said outer surface of said tubular body; said second polymeric liner being joined to said first liner through said stent openings to form at least one pocket about said solid segments; and
a bioactive agent located within said pocket about said solid segments of said tubular body.

28. A method of making an implantable composite device for delivery of bioactive agents associated therewith to a site of implantation of said device, said method comprising:
providing an implantable prosthetic stent having a generally cylindrical tubular body defined by solid segments and spaces therebetween, said tubular body defining an inner surface and an opposed outer surface;
applying a first polymeric liner to said inner surface;
applying a second polymeric liner to said outer surface;
joining said first and said second polymeric liners through said spaces between said stent segments to form a reservoir pocket adjacent to said solid segments; and
filling said reservoir pocket with a bioactive agent for delivery of said bioactive agent to the site of implantation of said device.

29. The method of claim 28, wherein said joining is at a location coextensive with said inner surface portion of said tubular body.

30. The method of claim 28, further comprising providing said first polymeric liner on an elongate mandrel.

31. The method of claim 30, further comprising disposing said tubular stent over said first polymeric liner.

32. The method of claim 31, further comprising disposing said second polymeric liner over said disposed tubular stent.

33. The method of claim 32, further comprising compressing said disposed second tubular polymer liner through said openings of said stent and into contact with said first tubular polymeric liner.

34. The method of claim 28, wherein said joining of said first and said second polymeric liners comprises adhering, laminating or bonding said second tubular polymeric liner to said first polymeric liner at a location substantially coextensive with said inner surface of said tubular body of said stent.

35. The method of claim 28, wherein said filling comprises applying a vacuum and pressure to said device after said pocket is formed to remove air from said pocket for replacement with said bioactive agent.

36. The method of claim 28, wherein said filling comprises first encapsulating said bioactive agent in a polymeric matrix and subsequently mixing said encapsulated bioactive agent with a fluid or gel for delivery thereof to said pocket.

37. The method of claim 28, wherein said filling comprises injecting said pocket with said bioactive agent.

38. The method of claim 28, wherein said filling comprises delivering said bioactive agent to said pocket by use of a mini-pump attached to said pocket.

39. The method of claim 28, wherein said filling is performed prior to implantation of said device.

40. The method of claim 28, wherein said filling is performed following implantation of said device.

41. A method of making an implantable composite device for delivery of bioactive agents associated therewith to a site of implantation of said device, said method comprising:
providing a first polymeric liner;
providing a second polymeric liner;
interposing an intermediate structural member between said first and second polymeric liners, said intermediate structural member being defined by solid segments and openings therebetween;
joining said first and second polymeric liners through said openings between said solid segments to form reservoir pockets adjacent to said solid segments; and filling said reservoir pockets with a bioactive agent for delivery of said bioactive agent to the site of implantation of said device.

42. A method for treating a lumen in a body, said method comprising:
inserting a generally cylindrical implantable composite device for delivery of bioactive agents incorporated therewith into said lumen, the device comprising a first polymeric liner; a second polymeric liner; an intermediate structural member interposed between said first and second liners, said intermediate structural member being defined by solid segments and openings therebetween such that the first liner can be bonded to the second liner through said openings to form at least one pocket about said solid segments; and a bioactive agent located within said pocket; and
affixing said implantable composite device to said lumen such that it will stay where positioned.

43. The method of claim 42, wherein said implantable composite device is inserted and/or accessed percutaneously.

44. The method of claim 42, wherein said implantable composite device is inserted and/or accessed using open or minimally invasive surgery.

45. The method of claim 42, wherein said bioactive agents are loaded into said pockets prior to or at the time of implant.

46. The method of claim 42, wherein said bioactive agents are loaded into said pockets after implant.

47. A method for treating a lumen in a body, said method comprising:
inserting an implantable composite device for delivery of bioactive agents incorporated therewith into said lumen, the device comprising an elongate stent having a generally cylindrical tubular body defined by solid segments and openings between said solid segments, said tubular body defining an inner surface and an opposed outer surface; a first polymer liner positioned about said inner surface of said tubular body; a second polymer liner positioned about said outer surface of said tubular body; said second polymer liner being joined to said first polymer liner through said stent openings to form a pocket about said solid segments; and a bioactive agent located within said pocket about said solid segments of said tubular body; and
affixing said implantable composite device to said lumen such that it will stay where positioned.
